(19) United States
(12) Patent Application Publication
(10) Pub. No.: US 2005/0243970 A1
(43) Pub. Date: Nov. 3, 2005

(54) X-ray mammography apparatus with radiation dose-reducing filter

(76) Inventor: Philipp Bernhardt, Forchheim (DE)

Correspondence Address: SCHIFF HARDIN, LLP;PATENT DEPARTMENT, 6600 SEARS TOWER, CHICAGO, IL, 60606-6473, US

(21) Appl. No.: 11/096,119

(22) Filed: Mar. 31, 2005

(30) Foreign Application Priority Data
Mar. 31, 2004 (DE) 10 2004 015 860.6

(51) Int. Cl. A61B 6/04; G21K 3/00

(52) U.S. Cl. 378/156; 378/37

(57) ABSTRACT

An x-ray apparatus for mammography, with an x-ray tube having a tungsten, a filter downstream in the beam direction of the x-ray tube and a detector downstream from the filter, the detector being produced from a semiconductor material. To improve the quality of mammographic x-ray exposures as well as to simultaneously reduce the radiation dose, the filter is produced from a filter material having a K-absorption edge in the range between 3.8 keV and 7.3 keV.

### BACKGROUND OF THE INVENTION

1. Field of the Invention

The invention concerns an x-ray apparatus for mammography of the type having a filter between the x-ray source and the radiation detector.

2. Description of the Prior Art

An x-ray apparatus of type above type is known from Flynn et al, “Optimal Radiographic Techniques for Digital Mammograms Obtained with an Amorphous Selenium Detector”, Proceedings of SPIE Vol. 5030 (2003). In this article, five randomly selected filter materials (Ag, Al, Mo, Rh and Sn) are compared based on computer simulations. Filters produced from Ag and Sn have proven to be advantageous. The radiation dose absorbed by a patient was smallest for Sn. Despite such a reduced radiation dose, mammographic examinations can cause radiation damage, such that a further reduction of the radiation dose is desirable.

### SUMMARY OF THE INVENTION

An object of the present invention is to provide an x-ray mammography apparatus that avoids the disadvantages exhibited by the prior art. Alternative filter materials should be specified with which a radiation dose absorbed by a patient can be further reduced with the same or an improved quality of the mammographic x-ray exposures.

This object is achieved in accordance with the invention by an x-ray mammography apparatus having a filter produced from a filter material with a K-absorption edge in the range between 3.8 keV and 7.3 keV. Such a filter is designated below as a K-filter. In general, x-ray radiation with quantum energies smaller than approximately 15 keV is strongly absorbed by tissue. On average, K-filters exhibit substantially higher atomic cross-sections for these quantum energies than for higher-energy quantum energies in the range from approximately 15-45 keV. The x-ray radiation strongly absorbed by the tissue and a radiation exposure associated therewith thus can be reduced.

Pathological variations in the tissue of a breast (such as, for example, calcifications or tumors) can be detected by means of mammography. Due to different absorption properties (such as, for example, density or thickness) the x-ray radiation must be adapted to the respective tissue. This is possible with the use of a K-filter. X-ray radiation transmitted through a K-filter with a maximal quantum energy less than 45 keV has a continuous spectrum with a maximum. By changing the maximum quantum energy or the thickness of the filter, the maximum too-high or too-low values can be shifted, in particular into a range between 15 and 45 keV that is advantageous for mammography. It is therewith possible to qualitatively optimize mammographic x-ray exposures. For example, the contrast and the signal-to-noise ratio can be maximized.

According to a further embodiment of the invention, the x-ray tube is operated with a peak voltage between 15 and 45 kVp. The x-ray radiation generated with this has a maximum quantum energy between 15 and 45 keV that is particularly advantageous for qualitatively high-grade mammographic x-ray exposures.

According to a further embodiment of the invention, the semiconductor material of the detector is produced from selenium. Such a detector is used in conventional x-ray apparatuses for mammography. It is commercially available.

In another embodiment, the filter material is selected from the following group: Ca, Sc, Ti, V, Cr, Mn, Fe. These filter materials particularly significantly absorb x-ray quanta with an energy smaller than approximately 15 keV. X-ray quanta with energies greater than approximately 15 keV are less significantly absorbed. The above filter materials have atomic numbers between 20 and 26. For these atomic numbers, absorption of x-ray radiation with an energy in the range of approximately 15-45 keV is primarily caused by a photo effect occurring in the filter material. The photo effect causes no scatter radiation that can contribute to a dose absorbed by a patient.

With the above filter materials, it is possible to monitor the radiation dose, for example via selection of a suitable absorption thickness of the filter. Furthermore, the x-ray radiation transmitted through the filter can be adapted to the absorption properties of the tissue, for example by a suitable selection of the maximum quantum energy. Mammographic x-ray exposures with improved quality can be produced using the aforementioned filter materials with a reduced radiation dose.

According to a further embodiment of the invention, the filter exhibits a thickness in the range of 0.05 to 1 mm in the beam direction. A radiation dose particularly suited for mammography can be generated with this thickness.

### DESCRIPTION OF THE DRAWINGS

FIG. 1 shows the effective atomic cross-sections of various elements suitable for use in the filter of the mammography apparatus of the interior.

FIG. 2a shows quality factors for microcalcifications.

FIG. 2b shows quality factors for tumors.

FIG. 3 shows intensity distributions for explaining the invention.

### DESCRIPTION OF THE PREFERRED EMBODIMENTS

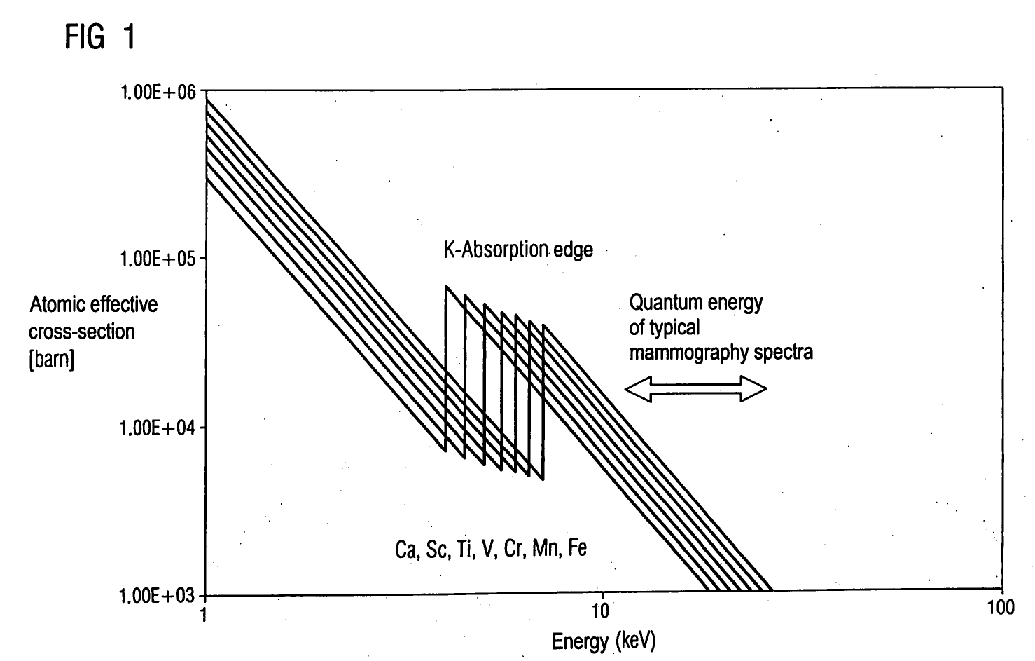

Effective atomic cross-sections of the elements Ca, Sc, Ti, V, Cr, Mn, Fe are shown in FIG. 1 with quantum energies in the range of 1 to approximately 45 keV. The range of quantum energies typical for mammography (from approximately 15 to 45 keV) is designated by the double arrow. The K-absorption edges are between 4.0 and 7.1 keV. The effective atomic cross-section increases by approximately one order of magnitude at the K-absorption edges. The atomic effective cross-sections are on average smaller for x-rays with quantum energies typical for mammography than for x-rays with quantum energies smaller than approximately 15 keV.

The elements shown in FIG. 1 particularly absorb quantum energies smaller than approximately 15 keV. Such quantum energies are significantly absorbed in tissue and significantly contribute to the radiation dose absorbed by a patient. These quantum energies can be largely suppressed by filtering. Quantum energies in the range between approximately 15 and 45 keV are particularly important for qualitatively good x-ray exposures in mammography. They are transmitted well through a filter produced from the above materials. It is thus possible to achieve a maximum quality of the x-ray exposures with minimum radiation dose.

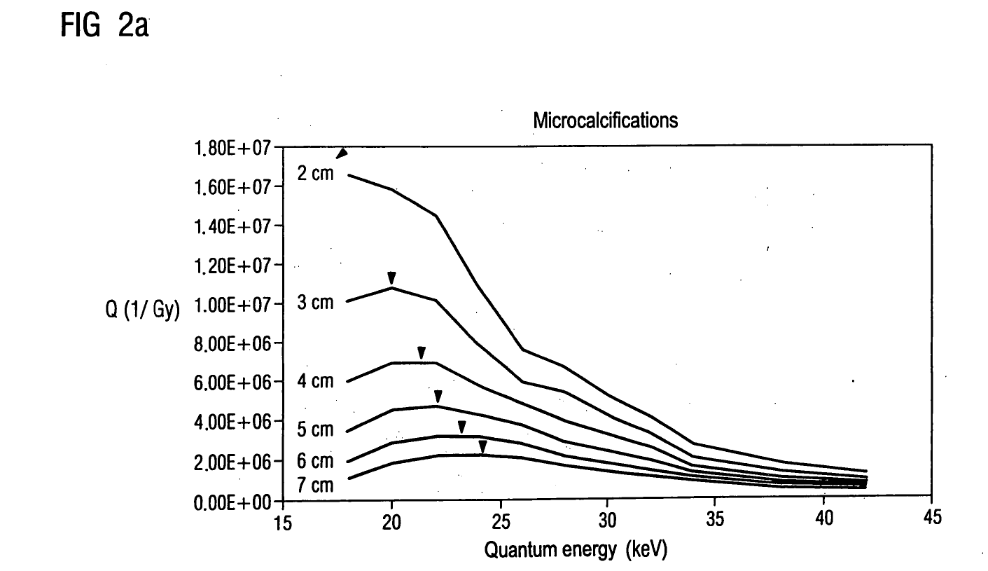

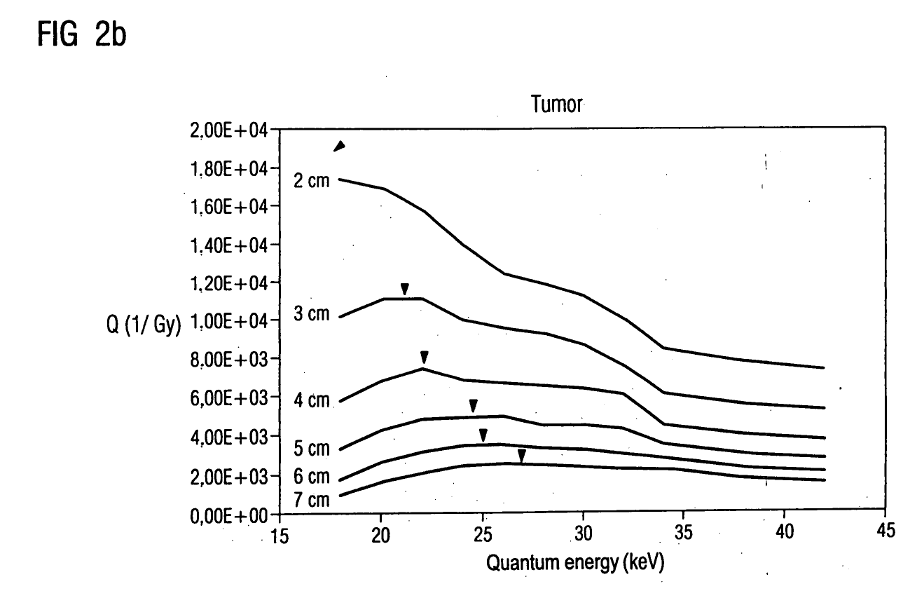

Simulated quality factors of mammographic x-ray exposures are plotted against quantum energies in FIGS. 2a and 2b. Tissue with thicknesses of 2, 3, 4, 5, 6 and 7 cm, with microcalcifications or tumors, are considered. The quantum energies lie in a range of 18 to 42 keV. The quality factors are typically defined as:
Q=(CNR)²/D
wherein CNR is defined as the contrast-to-noise ratio and D is an averaged radiation dose absorbed by the tissue. The quality factors have been simulated under the following suppositions:
the tissue is composed of 50% fatty tissue (Ap6) and 50% adenoidal tissue (BR12),
BR12 comprises 54.6% H; 36.9% C; 1.07% N; 7.1% O; 0.0251% Cl; 0.15% Ca with a density of 0.97 g/cm3,
AP6 comprises 53.7% H; 37.2% C; 1.09% N; 6.85% O; 1.04 F; 0.0256% Cl with a density of 0.92 g/cm3,
microcalcifications and tumors have a cylindrical shape with a central axis perpendicular to the x-ray radiation and are arranged centrally in the tissue,
microcalcifications are comprised of Ca5 P3 O13 H, exhibit a density of 3.2 g/cm3 and a radius of 0.1 mm,
tumors are comprised of BR12, exhibit a density of 1.044 g/cm3 and a radius of 2.5 mm,
the x-ray radiation possesses a focus of the size 0.3 mm,
the energy per x-ray exposure is 2.5 kWs,
x-ray radiation is pre-filtered with 1.0 mm Be,
the distance focus—detector is 650 mm,
the distance tissue—detector is 40 mm,
detection by means of an Se-detector with an absorption thickness of 0.25 mm, a density of 4.28 g/cm3, a pixel size of 0.07 mm, a fill factor of 1.00 and a pixel count of 4096×3584.

In FIGS. 2a and 2b, the maxima of the quality factors are characterized by triangles. D is minimal and CNR maximal at the quantum energy belonging to a maximum. For greater thickness of the tissue, the maximum lies at higher quantum energies. Furthermore, the maxima are dependent on whether calcifications or tumors are contained in the tissue. Given the same tissue thickness, the maximum for tumors lies at higher quantum energies than for calcifications. FIGS. 2a and 2b show that an adaptation of the x-ray radiation to conditions of the tissue is necessary for qualitatively high-grade mammographic x-ray exposures.

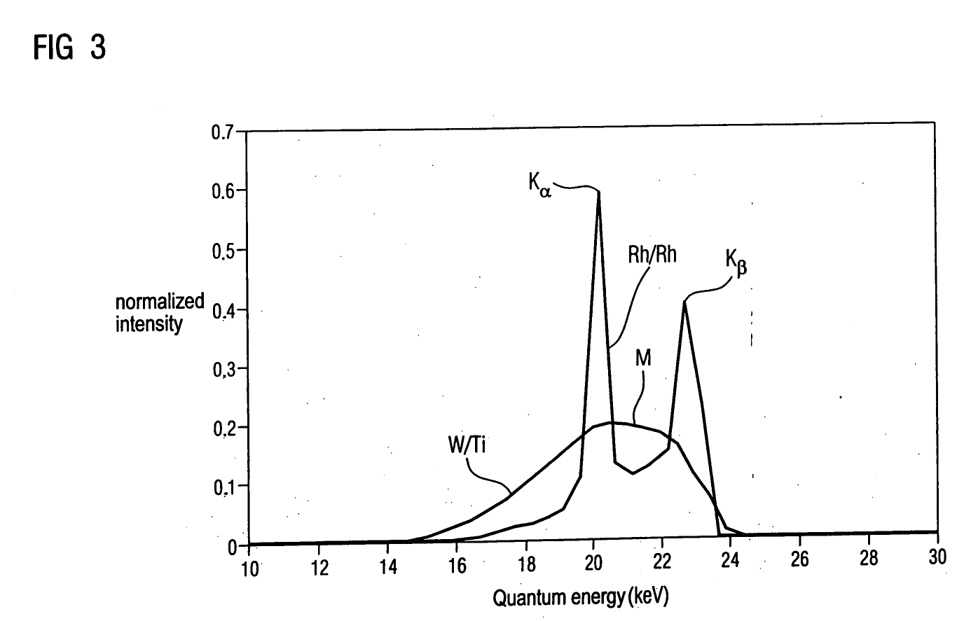

FIG. 3 shows normalized intensity distributions of x-ray radiation. These have been calculated according to J. Boone, “Molybdenium, rhodium and tungsten anode spectral models using interpolating polynomials with application to mammography”, Med. Phys. 24(12), 1863-1874 (1997). W/Ti designates an intensity distribution given use of a tungsten anode in combination with a 0.4 mm thick filter produced from Ti and given a peak voltage of 24 kVp. Rh/Rh designates an intensity distribution for a combination made of an anode produced from Rh and an 0.18 mm-thick filter produced from Rh, given a peak voltage of 34 kVp.

Rh/Rh is affected by two characteristic x-ray lines at approximately 20.0 keV K_{α} and 23.2 keV K_{β}. In contrast to this, W/Ti is continuously designed [sic] and exhibits a maximum M at a quantum energy of approximately 21 keV that is optimal for the detection of calcifications. While given Rh/Rh the characteristic x-ray lines K_{α} and K_{β} are invariable with regard to their position, given W/Ti the maximum M can be shifted towards smaller or larger quantum energies by changing the peak voltage or via selection of a suitable thickness of the filter. Given an optimal intensity distribution adapted to a tissue to be examined, the maximum M of the respective quantum energy lies where the quality factors according to FIG. 2a or 2b are maximal. The intensity distribution generated with the W/Ti system can be flexibly adapted, such that larger quality factors can be achieved.

The W/Ti system is particularly advantageous for quantum energies>approximately 25 keV. For example, with CNR=1.00 the radiation dose can therewith be reduced by 44% relative to an Rh/Rh system in the detection of a tumor.

Although modifications and changes may be suggested by those skilled in the art, it is the intention of the inventor to embody within the patent warranted hereon all changes and modifications as reasonably and properly come within the scope of his contribution to the art.

1. In an x-ray mammography apparatus having an x-ray tube with a tungsten anode, a radiation detector, and a filter disposed between said x-ray tube and said radiation detector in a region permeated by x-rays when said x-ray tube emits an x-ray beam, the improvement of said filter comprising:
a filter material having a K-absorption edge in a range between 3.8 keV and 7.3 keV.

2. The improvement of claim 1 wherein said x-ray apparatus includes an operating unit for operating said x-ray tube, and wherein said improvement comprises said operating unit operating said x-ray tube at a peak voltage between 15 and 45 kVp.

3. The improvement of claim 1 comprising said detector comprising a selenium-based semiconductor material.

4. The improvement of claim 1 wherein said filter material is selected from the group consisting of Ca, Sc, Ti, V, Cr, Mn and Fe.

5. The improvement of claim 1 comprising said filter having a thickness in a direction of propagation of said x-rays in a range between 0.05 mm and 1 mm.
